(19) United States
(12) Patent Application Publication
(10) Pub. No.: US 2013/0342811 A1
(43) Pub. Date: Dec. 26, 2013

(54) APPARATUS FOR MEASURING OPTICAL PROPERTIES OF AN OBJECT

(71) Applicants: Berndt Warm, Schwaig (DE); Stefan Schmid, Heilsbronn (DE); Claudia Gorschboth, Nurnberg (DE); Christof Donitzky, Eckental/Eschenau (DE)

(72) Inventors: Berndt Warm, Schwaig (DE); Stefan Schmid, Heilsbronn (DE); Claudia Gorschboth, Nurnberg (DE); Christof Donitzky, Eckental/Eschenau (DE)

(73) Assignee: WaveLight GmbH, Erlangen (DE)

(21) Appl. No.: 13/983,939

(22) PCT Filed: Feb. 15, 2011
(86) PCT No.: PCT/EP2011/000713

§ 371 Date: Sep. 9, 2013

(51) Int. Cl. A61B 3/10; G01B 9/02

(52) U.S. Cl. CPC A61B 3/102; G01B 9/02091
USPC 351/206; 356/479

(57) ABSTRACT

An apparatus for measuring optical properties of an object—such as, in particular, an eye—comprises a wavefront sensor for surveying wavefront aberrations generated by the object and an optical coherence tomograph, so that both wavefront aberrations and structures of the object can be surveyed. For this purpose a broadband laser radiation-source is provided for the OCT. A reference beam is generated with a retroreflector, and a beam-splitter serves as optical component both for the wavefront determination and for the OCT.

### TECHNICAL FIELD

The invention relates to an apparatus for measuring optical properties of an object.

### BACKGROUND

In application of the invention, in particular the human eye enters into consideration by way of object to be surveyed. In the following, the invention will be elucidated with regard to the measurement of optical properties of the eye.

The surveying of the optical properties of the eye is fundamental for refractive operations—that is to say, surgical interventions in respect of the eye for the purpose of altering the refractive power thereof in order to cure or alleviate visual disturbances. A widely known ophthalmological intervention of this type is LASIK. In this case, corneal tissue is ablated in targeted manner by laser radiation in order to improve the imaging properties of the eye. It has become evident that the resection of material that is required for the improvement of the visual acuity of the patient can be determined with good results with so-called ray tracing. In the case of ray tracing—that is to say, the mathematical back-tracing of ray paths through the eye—an optimal ablation profile that is to say, a preset for the resection of the corneal tissue—is computed by optimisation of the ray trajectories.

Extensive measurements in respect of the eye are required for this; in particular, the parameters constituted by wavefront, topography of the outer and inner surfaces of the cornea, outer and inner surfaces of the lens, as well as the optical lengths in the eye, have to be determined, in order to obtain good outcomes for the visual acuity of the patient after the refractive operation.

### SUMMARY OF EXAMPLE EMBODIMENTS

The object underlying the invention is to make available an apparatus with which the optical properties of an object—such as, in particular, an eye can be determined quickly and comprehensively.

For this purpose the invention provides an apparatus in which a wavefront sensor and an optical coherence tomograph are integrated.

By an ‘optical coherence tomograph’ in the sense of the invention, an apparatus for optical coherence tomography is to be understood.

Wavefront sensors as such are known in the state of the art; in particular, they operate in accordance with the Tscherning principle, in accordance with the Hartmann-Shack principle, or in accordance with the curvature-sensor principle.

Instruments for optical coherence tomography (OCT) are also known as such, it being possible for the OCT to be realised in different ways; in particular, a distinction is made between time-domain OCT and frequency-domain OCT.

In particular, a finding underlying the present invention is that wavefront sensors and optical coherence tomographs can be combined with one another in very advantageous manner, whereby not only instrumental components both for the wavefront determination and for the optical coherence tomography are capable of being employed jointly but also, at the same time, a plurality of parameters required for the ray tracing elucidated above can be ascertained very quickly with high precision without the patient having to be confronted with different measuring systems. With OCT, in particular determinations of length can be carried out on and in the eye.

Moreover, a finding underlying the invention is that by virtue of the integration—described above—of wavefront determination and optical coherence tomography a plurality of optical parameters, complementing one another optimally, of the object to be surveyed can be acquired, in particular for the aforementioned ray tracing, for which all the requisite determinants can be ascertained in virtually a single measuring procedure. The term ‘measuring’ here encompasses both the quantitative determination of a magnitude and the relative determination thereof.

The invention makes it possible to employ one and the same common radiation-source both for the wavefront sensor and for the optical coherence tomograph.

Another variant of the invention provides that optical components of the apparatus are employed both for radiation bundles of the wavefront sensor and for radiation bundles of the optical coherence tomograph. This not only reduces the instrumental complexity but also facilitates the alignments and enhances the accuracy of measurement as well as the compatibility of the results of measurement acquired with both systems.

A broadband laser that is suitable for optical coherence tomography, a broadband LED or a superluminescent diode is preferably employed by way of common radiation-source.

### BRIEF DESCRIPTION OF THE DRAWINGS

In the following, embodiments of the invention will be elucidated in more detail on the basis of the drawings.

Shown are:

FIG. 1 schematically for the purpose of elucidation, a wavefront sensor according to the Tscherning principle;

FIG. 2 an apparatus in which a wavefront sensor according to the Tscherning principle and a device for optical coherence tomography are integrated;

FIG. 3 a modification of the apparatus according to FIG. 2, with two detector systems;

FIG. 4 schematically for the purpose of elucidation, a wavefront sensor according to the Hartmann-Shack principle;

FIG. 5 an apparatus in which a wavefront sensor according to the Hartmann-Shack principle and a device for optical coherence tomography are integrated; and

FIG. 6 an apparatus in which a wavefront sensor according to the curvature-sensor principle and a device for optical coherence tomography are integrated.

### DETAILED DESCRIPTION OF THE DRAWINGS

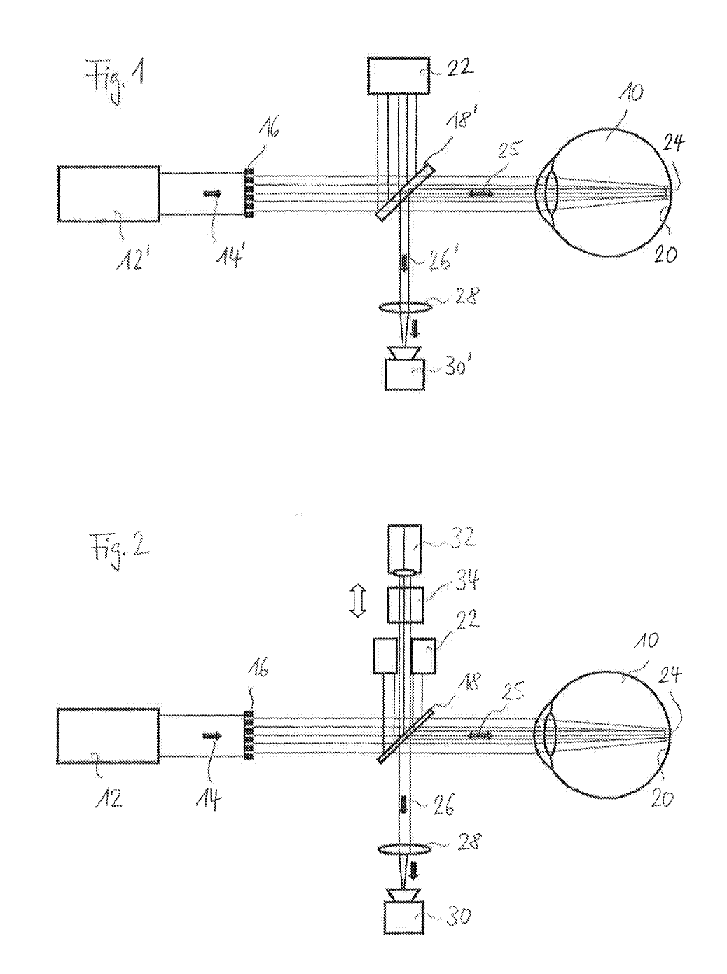

Wavefront sensors according to Tscherning are well-known to a person skilled in the art. According to FIG. 1, optical properties of the overall optical system constituted by the eye 10 can be determined with such a wavefront sensor. Ordinarily in this connection, radiation 14′ generated by a laser 12′ is split up via an aperture mask 16 into a plurality of partial beams which strike the eye 10 in parallel and generate on the retina 20 of the eye an image 24 in the form of individual dots, corresponding to the partial beams. In the process the radiation passes through a beam-splitter 18′. Radiation reflected on the surface of said beam-splitter and not used any further arrives at a beam trap 22.

The image generated on the retina 20 in the form of a pattern of dots is contained in the radiation 25 coming from the eye 10 and is projected into a camera 30′ via the beam-splitter 18 (FIG. 1, radiation 26′) and imaging optics 28. From the deviations of the positions of the individual dots from set position values, optical imaging errors of the eye are determined in a manner known as such.

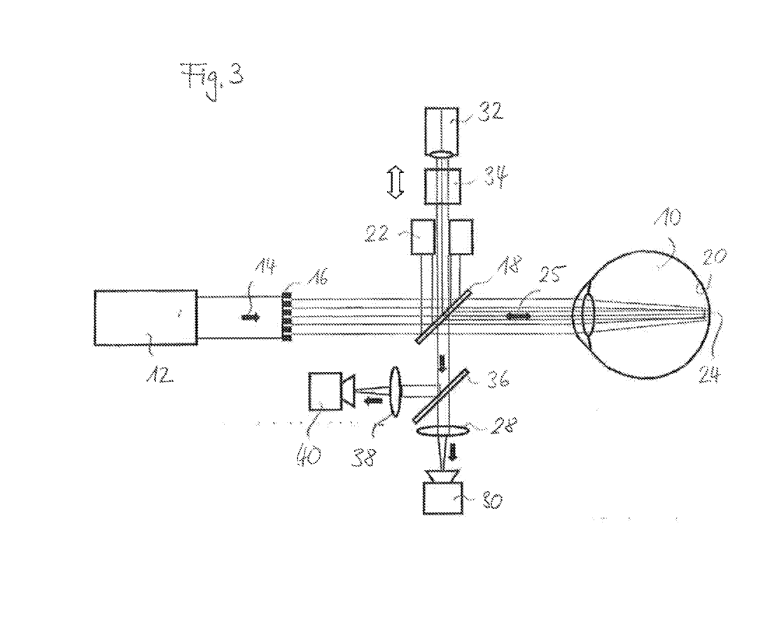

FIG. 2 shows an integration, according to the invention, of an optical coherence tomograph into a wavefront sensor according to FIG. 1. A spectrally broadband laser source, which is designed in such a way that an OCT is capable of being implemented with it, now serves as radiation-source. With the beam-splitter 18 a reference beam required for the OCT is generated in the form of a partial beam (which in FIG. 2 is deflected upwards). The partial beam is widened to the diameter of the beam that comes out of the eye. If the OCT is implemented in accordance with the time-domain process, the optical path-length of the reference beam has to be altered. This can be done, for example, by controlled mechanical movement of the retroreflector 32 or, for example, through the use of a path-length changer, such as, for example, rotating prisms, mirrors or such like.

Radiation 25 coming from the eye 10 is deflected downwards in FIG. 2 in the direction of the arrow 26 via the beam-splitter 18, and arrives at a detector 30 via optics 28. The beam coming from the retroreflector 32 also passes through the beam-splitter 18 and arrives at the detector 30 by way of reference beam. In the detector system 30 the reference beam and the measuring beam coming from the eye are superimposed. The reference beam generates a background, and the reflections coming from the eye are superimposed on this background. If the reference beam and the reflection are incoherent, the image generated in the detector 30 is capable of being evaluated in conventional manner. If the differences in optical path-length between reference beam and reflections are very small, the superimposing beams are coherent, and interference phenomena occur in the detector, being evaluated in a manner known as such for optical coherence tomography.

Likewise, in this embodiment of the invention the pattern of dots coming from the eye 10 and described above can be recorded with the detector 30 and evaluated in accordance with the Tscherning principle in a manner known as such, in order to determine wavefront aberrations that were generated by the optical system constituted by the eye.

By way of detector 30, cameras known for this purpose may be employed, but for short measuring-times fast detectors should be provided, such as high-speed cameras, photodiodes with respectively assigned preamplifiers, or other arrays of detectors.

If the OCT is implemented in accordance with the so-called Fourier domain, arrays of detectors known for this purpose may be employed in combination with a dispersive element (prism, grating).

FIG. 3 shows a modification of the apparatus according to FIG. 2, to the effect that, in addition to the detector 30, a further high-speed detector 40 is employed. A beam-splitter 36 couples a partial radiation out of the radiation 25 coming from the eye, and via optics 38 said partial radiation arrives at the high-speed detector 40 for the implementation of the OCT. Instead of the beam-splitter, a folding mirror may also be provided. With the use of different detectors for the ascertainment of the wavefront aberration, on the one hand, and for the OCT, on the other hand, it is possible to obtain a high two-dimensional local resolution for the wavefront measurement in targeted manner, whereas for the optical coherence tomography a rapid evaluation of the signal with a detector that is suitable for this purpose is made possible.

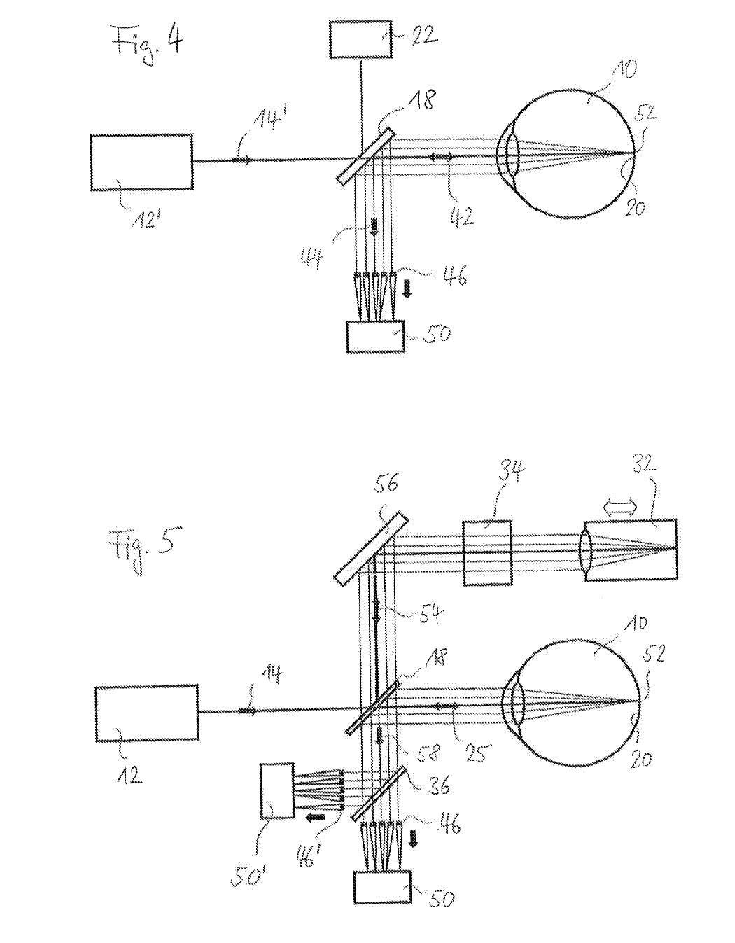

FIG. 4 shows schematically a wavefront sensor operating in accordance with the Hartmann-Shack principle. In all the Figures, components that correspond to one another or that are functionally similar are provided with the same reference symbols. In the case of the Hartmann-Shack principle, the retina 20 of the eye 10 is illuminated with a punctiform laser beam 14′ from a laser 12′. The light scattered on the retina 20 emerges from the eye 10 in the form of a distinctly wider radiation bundle. This radiation bundle 42 is deflected downwards in FIG. 4 (arrow 44) by the beam-splitter 18 and is then broken down via a lens array 46 into partial beams which are focused onto a CCD detector 50. The image is a pattern of dots. In the case of a wavefront without aberration, a set pattern of dots arises on the detector. If a real eye is surveyed, as a rule the dots of the image are not situated exactly at the positions of the set pattern of the dots. From the deviations of the imaged dots from the set dots, the curvature of the wavefront is determined in a manner known as such, and from this the optical properties of the eye are then inferred.

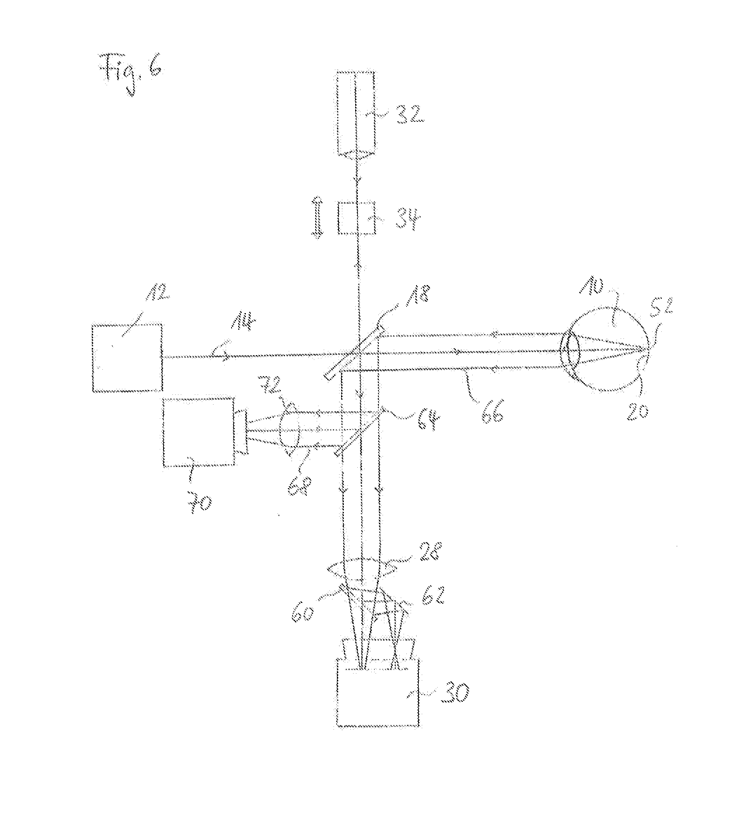

FIG. 5 shows the linkage of an optical coherence tomograph with a wavefront sensor according to FIG. 4. For this purpose a spectrally broadband laser radiation-source 12 is employed as a common radiation-source for the wavefront sensor and for the optical coherence tomograph. The reference beam required for the OCT is generated with the beam-splitter 18 (FIG. 5, reference symbol 54). The retroreflector 32 widens the reference beam reflected on it. An array of mirrors, described in more detail further below, or a deformable mirror 56 is arranged in the beam path of the reference beam.

As in the case of the embodiment according to FIGS. 2 and 3, the alteration of path-length can be carried out, for example, with a path-length changer 34 or by mechanical movement of the retroreflector 32 (in the case of time domain).

A lens array 46 splits up the radiation 58 coming from the eye and generates individual dots in the detector 50. The reference beam required for the OCT arises from a plane wavefront and therefore impinges on other points of the detector, so that no interference between the beams takes place. In order to enable interference, the aforementioned array of mirrors or a deformable mirror 56 is provided. For example, mirrors are known that are assembled in the form of an array from individually addressable individual mirrors (MEMS), or deformable mirrors are also known with which radiation can be controlled. The array of mirrors or the deformable mirror 56 is controlled in such a way that the superposition of measuring beam and reference beam that is necessary for an interference takes place on the detector 50.

Corresponding to the embodiment according to FIG. 3, elucidated above, also in the case of the apparatus according to FIG. 5 partial radiation can be directed onto a second detector system 50′ by means of a beam-splitter 36. The aforementioned detectors also enter into consideration here by way of detector systems.

FIG. 6 shows a further variant of the invention, in which the curvature-sensor principle, known as such, is employed for the wavefront sensor. Here (just as in the embodiments described above) an LED or an SLD (superluminescent diode) may be employed by way of radiation-source. A collimated light beam 14 is generated and is guided onto the retina. The light back-scattered on the retina emerges from the eye in the form of a wider radiation bundle. In the embodiment of a curvature sensor that is shown, the radiation bundle impinges on a beam-splitter 60 after passing through focusing optics 28. The light transmitted by the beam-splitter 60 impinges directly on the detector arrangement of the camera. The light reflected on the beam-splitter 60 is directed onto the camera detectors in temporally offset manner via a further deflection on the mirror 62 and hence via a longer optical path. The optical path for the radiation transmitted by the beam-splitter 60 is preferentially shorter than the back focal length of the focusing optics. For the reflected radiation portion which is deflected via the mirror 62 the optical path-length is preferentially longer than the back focal length, this also being indicated schematically in FIG. 6. The wavefront can then be ascertained, in a manner known as such, from a point-to-point contrast of the two recorded intensities. FIG. 6 shows, furthermore, the indication of an optical coherence tomograph with the components, already elucidated above, constituted by retroreflector 32 and path-length changer 34 for the reference beam (also called reference arm). In the embodiment a broadband laser 12 serves as radiation-source for the OCT. A beam-splitter 64 couples radiation portions 68 out of the radiation coming from the eye 10, and these radiation portions are projected via optics 72 into a high-speed detector 70 for the OCT. As in the above examples, also in this embodiment the time domain is employed for the OCT, and the modifications elucidated above on the basis of the other embodiments may likewise be employed here analogously.

The determinations of optical properties of an object that are possible with the apparatuses that have been described are not only of use for refractive surgery in respect of the eye but may also serve for the computation of intraocular lenses, for cataract diagnosis, fundus examination and for the construction of refractometers.

1. Apparatus for measuring optical properties of an object, comprising:
a wavefront sensor with a radiation-source, with means for directing radiation from the radiation-source onto the object in such a manner that the radiation transirradiates the object, and with a detector for detecting radiation coming from the object for the purpose of detecting wavefront aberrations generated by the object, characterised by
an optical coherence tomograph with a radiation-source and with means for directing a radiation measuring arm from the radiation-source onto the object and with a detector for detecting radiation, reflected from the object.

2. Apparatus according to claim 1, characterised in that the wavefront sensor and the optical coherence tomograph have the same common radiation-source.

3. Apparatus according to claims 1, characterised in that said means of the wavefront sensor for directing radiation and said means of the optical coherence tomograph for directing the radiation measuring arm onto the object are at least partly identical.

4. Apparatus according to claim 2, characterised in that the common radiation-source is a broadband laser adapted for optical coherence tomography or a broadband LED or a superluminescent diode or a supercontinuum source.

5. Apparatus according to claim 1, characterised in that the object is an eye.

6. Apparatus according to claim 1, characterised in that the wavefront sensor is a Tscherning aberrometer.

7. Apparatus according to claim 1, characterised in that the wavefront sensor is a Hartmann-Shack sensor.

8. Apparatus according to claim 1, characterised in that the wavefront sensor is a curvature sensor.

9. Apparatus according to claim 1, characterised in that the wavefront sensor is a digital wavefront sensor, in particular a digital wavefront camera.

10. Apparatus according to claim 1, characterised in that the optical coherence tomograph has been adapted for, in particular, topographical measurements or length measurements.

11. Apparatus according to claim 1, characterised in that the wavefront sensor and the optical coherence tomograph utilise partly identical ray bundles.
